SEQUENCE LISTING (3 sequences)

SEQ ID NO: 1 - protein, 214 aa, Artificial sequence
  light chain of ox40L antibody
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial sequence
  heavy chain of Mab1
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 450 aa, Artificial sequence
  heavy chain of Mab2
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2012/0128687 A1
(43) Pub. Date: May 24, 2012

(54) NOVEL ANTIBODY FORMULATION

(75) Inventors: Michael Adler, Riehen (CH); Christine Wurth, Loerrach (DE)

(73) Assignee: Hoffmann-La Roche.Inc., Nutley, NJ (US)

(21) Appl. No.: 13/168,635

(22) Filed: Jun. 24, 2011

(30) Foreign Application Priority Data
Jun. 25, 2010 (EP) 10167357.2

(51) Int. Cl. A61K 39/395; A61P 29/00; A61P 11/06

(52) U.S. Cl. 424/158.1

(57) ABSTRACT

Pharmaceutical liquid formulation of an antibody against human OX40L, a process for the preparation and uses of the formulation.

### CROSS REFERENCE TO RELATED APPLICATIONS

This application claims the benefit of European Patent Application No. 10167357.2, filed on Jun. 25, 2010, the disclosure of which is incorporated herein by reference in its entirety.

### FIELD OF THE INVENTION

The present invention relates to a pharmaceutical liquid formulation of an antibody against human OX40L, a process for the preparation and uses of the formulation.

### BACKGROUND

An ongoing challenge in the clinical development of therapeutic antibodies is the development of liquid formulations providing a stable environment for storage and administration of these antibodies. Stable liquid formulations of therapeutic antibodies are particularly difficult to obtain when the formulation includes antibodies present at high concentrations.

Antibodies against OX40L are of therapeutic interest, particularly in the treatment of inflammatory diseases. Human antibodies against human OX40 ligand (OX40L, gp34, Swiss Prot P23510) are described in WO2006/029879. These antibodies inhibit the interaction of OX40L with OX40 in an ELISA using immobilized OX40L at a coating concentration of 0.5 μg/ml with an IC50 value of no more than 4 nM.

Low concentration formulations, in liquid form, lyophilized form or in liquid form reconstituted from a lyophilized form, of antibodies against OX40L are disclosed in WO2009/141239.

### SUMMARY

One aspect of the invention provides for a pharmaceutical liquid formulation comprising:
1 to 200 mg/mL of an antibody against OX40L;
1 to 100 mM of a buffer selected from histidine acetate and sodium acetate;
0.001 to 1% of a surfactant;
(a) 5 to 500 mM of a stabilizer; or
(b) 5 to 500 mM of a stabilizer and 5 to 500 mM of a tonicity agent; or
(c) 5 to 500 mM of a tonicity agent;

at a pH in the range of from 4.0 to 7.0. In one embodiment, the formulation comprises

1 to 100 mM of a buffer selected from histidine acetate buffer at pH 5.8 and sodium acetate-buffer at pH 5.3;

(a) 5 to 500 mM of a stabilizer selected from methionine, trehalose and arginine HCl; or

(b) 5 to 500 mM of methionine as a stabilizer and 5 to 500 mM of a tonicity agent selected from trehalose and arginine HCl; or

(c) 5 to 500 mM of a tonicity agent selected from trehalose and arginine HCl.

In one embodiment, the formulation comprises 20 mM of a buffer selected from sodium acetate-buffer at pH 5.3 and histidine acetate buffer at pH 5.8;

(a) a stabilizer selected from 10 mM methionine, 200 mM trehalose and 130 mM arginine HCl; or

(b) 10 mM methionine as a stabilizer and a tonicity agent selected from 200 mM trehalose and 130 mM arginine HCl; or

(c) a tonicity agent selected from 200 mM trehalose and 130 mM arginine HCl. In one embodiment, the formulation comprises a surfactant selected from the group consisting of polysorbate 20 (sold under the trademark Tween 20™), polysorbate 80 (sold under the trademark Tween 20™) and a polyethylene-polypropylene copolymer sold under the name Poloxamer 188™.

In one embodiment, the formulation comprises a stabilizer and a tonicity agent wherein the stabilizer is selected from the group consisting of sugars and amino acids, and the tonicity agent is selected from the group consisting of sodium chloride, amino acids and sugars. In certain embodiments, the stabilizer is selected from the group consisting of sucrose, trehalose, raffinose, arginine, glycine, histidine, tryptophane and methionine, and the tonicity agent is selected from the group consisting of trehalose and arginine. In one embodiment of the formulation, the stabilizer is methionine in an amount of 10 mM; and the tonicity agent is selected from the group consisting of trehalose in an amount of about 200 mM and arginine in an amount of about 130 mM.

In one embodiment, the formulation comprises a tonicity agent selected from the group consisting of amino acids and sugars. In one embodiment, the tonicity agent is trehalose in an amount of 200 mM. In another embodiment, the tonicity agent is arginine in an amount of 130 mM.

In one embodiment, the formulation comprises histidine acetate in an amount of 20 mM, at pH 5.8. In another embodiment, the formulation comprises sodium acetate in an amount of 20 mM, at pH 5.3.

In certain embodiments, the antibody against human OX40L is a human antibody against human OX40L. in one embodiment, the antibody against human OX40L is a human antibody against human OX40L comprising the light chain of SEQ ID NO:1 and the heavy chain of SEQ ID NO:2 or 3.

Another aspect of the invention provides a pharmaceutical liquid formulation comprising approximately 150 mg/ml of a human antibody against human OX40L comprising the light chain of SEQ ID NO:1 and the heavy chain of SEQ ID NO:2 or 3; and

(i) 20 mM sodium acetate-buffer at pH 5.3;
0.05% of a surfactant;
10 mM methionine; and
200 mM trehalose; or

(ii) 20 mM of histidine acetate buffer at pH 5.8;
0.05% of a surfactant;
10 mM methionine; and
200 mM trehalose.

In one embodiment, the surfactant is Polysorbate 20 or Poloxamer 188.

Another aspect of the invention provides for use of the pharmaceutical liquid formulation in the therapeutic treatment of an inflammatory disease in a mammal.

Another aspect of the invention provides for a method for the therapeutic and/or prophylactic treatment of an inflammatory disease, particularly for the treatment of asthma, which method comprises administering a liquid formulation according to the invention.

### DETAILED DESCRIPTION OF THE EMBODIMENTS OF THE INVENTION

The present invention provides a pharmaceutical liquid formulation comprising an antibody against human OX40L.

The term “liquid” as used herein in connection with the formulation according to the invention denotes a formulation which is liquid at a temperature of at least about 2 to about 8° C. under atmospheric pressure.

The term “antibody against human OX40L” as used herein denotes an antibody that binds to human OX40 ligand (OX40L, gp34, Swiss Prot P23510). In one embodiment, the antibody against human OX40L comprises as light chain the light chain of SEQ ID NO:1 and as heavy chain the heavy chain of SEQ ID NO:2 (further named Mab1) or as light chain the light chain of SEQ ID NO:1 and as heavy chain the heavy chain of SEQ ID NO:3 (further named Mab2). In one embodiment, the antibody is an IgG1 kappa type antibody. In one embodiment, the antibody is a human or humanized antibody. In one embodiment, the antibody is a human antibody. In one embodiment, the antibody according to the invention binds to OX40L with a KD value of 10⁻¹² to 10⁻⁹ M in a BIAcore assay. Exemplary conditions are given in WO/2006/029879 and as follows: Instrument: Biacore 3000, running and reaction buffer: HBS-P (10 mM HEPES, 150 mM NaCl, 0.005% Tween 20, ph 7.4), 25° C. In the BIAcore assay the antibody is bound to a surface and binding of OX40L is measured by Surface Plasmon Resonance (SPR). The affinity of the binding is defined by the terms ka (rate constant for the association of the antibody to the antigen), kd (rate constant for the dissociation), and KQ (kd/ka).

The term “buffer” as used herein denotes a pharmaceutically acceptable excipient, which stabilizes the pH of a pharmaceutical preparation. Suitable buffers are well known in the art and can be found in the literature. Examples of pharmaceutically acceptable buffers are histidine-buffers, citrate-buffers, acetate-buffers, arginine-buffers or mixtures thereof, e.g. L-histidine or mixtures of L-histidine and L-histidine hydrochloride with pH adjustment with an acid or a base known in the art. Independently from the buffer used, the pH may be adjusted at a value in the range of from about 4.0 to about 7.0, e.g. from about 5.0 to about 6.0 or from about 5.5 to about 6.5, e.g. pH 5.3 or pH 5.8, with an acid or a base known in the art, e.g. hydrochloric acid, acetic acid, and citric acid and sodium hydroxide.

The term “surfactant” as used herein denotes a pharmaceutically acceptable excipient which is used to protect protein formulations against mechanical stresses like agitation and shearing. Examples of pharmaceutically acceptable surfactants include polyoxyethylensorbitan fatty acid esters (Tween) and polyoxyethylene-polyoxypropylene copolymer (Poloxamer, Pluronic). Examples of polyoxyethylenesorbitan-fatty acid esters are polysorbate 20 (sold under the trademark Tween 20™) and polysorbate 80 (sold under the trademark Tween 80™). Examples of polyethylene-polypropylene copolymers are those sold under the names Pluronic® F68 or Poloxamer 188™. Examples of polyoxy-ethylene alkyl ethers are those sold under the trademark Brij™

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit. Rev Ther Drug Carrier Syst 10(4):307-77, Wang (1999) Int J Pharm 185(2):129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-β-cyclodextrine, sulfobutylethyl-β-cyclodextrin, β-cyclodextrin, salts, e.g. sodium chloride, chelators, e.g. EDTA as hereafter defined.

The term “sugar” as used herein denotes an oligosaccharide. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides oligosaccharides are water soluble. Examples of oligosaccharides include sucrose, trehalose and raffinose.

The term “amino acid” as used herein denotes a pharmaceutically acceptable organic molecule possessing an amino moiety located at α-position to a carboxylic group. Examples of amino acids include arginine, glycine, histidine, tryptophane and methionine.

The term “polyols” as used herein denotes pharmaceutically acceptable alcohols with more than one hydroxy group. Suitable polyols comprise but are not limited to mannitol, sorbitol, and combinations thereof.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, glutathione, cysteine, methionine, citric acid, EDTA.

The term “tonicity agents” as used herein denotes pharmaceutically acceptable tonicity agents. Tonicity agents are used to modulate the tonicity of the formulation. The formulation can be hypotonic, isotonic or hypertonic. Isotonicity in general relates to the osmotic pressure relative of a solution usually relative to that of human blood serum. The formulation according to the invention may be hypotonic, isotonic or hypertonic, e.g. be isotonic. Suitable tonicity agents comprise but are not limited to sodium chloride, and any component from the group of amino acids and sugars.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines and salts, e.g. sodium chloride. An example for a sugar which can at the same time be a stabilizer and a tonicity agent is trehalose.

The compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives comprise but are not limited to ethanol, benzyl alcohol, phenol, m-cresol, p-chlor-m-cresol, methyl or propyl parabens, benzalkonium chloride.

The abovementioned buffers are generally used in an amount of about 1 mM to about 100 mM, for example in an amount of about 5 mM to about 50 mM or in an amount of about 10 mM to 20 mM.

When polysorbate 20 (Tween 20™) and polysorbate 80 (Tween 80™) are used as surfactants they are generally used in a concentration range of about 0.005 to about 0.2% or of about 0.01% to about 0.1% w/v (weight/volume).

The stabilizers may be present in the formulation in an amount of about 10 to about 500 mM, e.g. in an amount of about 10 to about 300 mM or in an amount of about 100 mM to about 300 mM.

Amino acids are generally used in an amount of about 10 to 500 mM, e.g. in an amount of about 10 to about 300 mM or in an amount of about 100 to about 300 mM.

Polyols can be used in an amount of about 10 mM to about 500 mM, e.g. in an amount of about 10 to about 300 mM or in an amount of about 100 to about 300 mM.

Antioxidants can be used in an amount of about 1 to about 100 mM, e.g., in an amount of about 5 to about 50 mM or in an amount of about 5 to about 20 mM.

Preservatives may generally be used in an amount of about 0.001 to about 2% (w/v).

One aspect of the invention relates to a pharmaceutical liquid formulation of an antibody against human OX40L comprising a buffer selected from histidine acetate and sodium acetate. In one embodiment, the pharmaceutical liquid formulation of an antibody against human OX40L comprises histidine acetate in an amount of 20 mM, at pH 5.8. In another embodiment, the pharmaceutical liquid formulation of an antibody against human OX40L comprises sodium acetate in an amount of 20 mM, at pH 5.3.

One aspect of the invention relates to a pharmaceutical liquid formulation of an antibody against human OX40L comprising a surfactant selected from polysorbate 20 (sold under the trademark Tween 20™), polysorbate 80 (sold under the trademark Tween 80™) and a polyethylene-polypropylene copolymer sold under the name Poloxamer 188™. In one embodiment, the invention relates to a pharmaceutical liquid formulation of an antibody against human OX40L comprising a surfactant selected from polysorbate 20 (sold under the trademark Tween 20™) and a polyethylene-polypropylene copolymer sold under the name Poloxamer 188™. In one embodiment, the invention relates to a pharmaceutical liquid formulation of an antibody binding to human OX40L comprising polysorbate 20 (sold under the trademark Tween 20™) in a concentration range of about 0.005 to about 0.2% or of about 0.01% to about 0.1% w/v (weight/volume).

One aspect of the invention relates to a pharmaceutical liquid formulation of an antibody against human OX40L comprising a stabilizer selected from the group consisting of sugars and amino acids, e.g. selected from sucrose, arginine, trehalose and raffinose, e.g. from sucrose, arginine and trehalose, e.g. trehalose in an amount of about 200 mM or arginine in the amount of 130 mM.

Another aspect of the invention relates to a pharmaceutical liquid formulation of an antibody against human OX40L comprising a stabilizer and a tonicity agent wherein the stabilizer is selected from the group consisting of sugars and amino acids; and the tonicity agent is selected from the group consisting of sodium chloride, amino acids and sugars.

Another aspect of the invention relates to a pharmaceutical liquid formulation of an antibody against human OX40L comprising a stabilizer and a tonicity agent wherein the stabilizer is a sugar, e.g. selected from sucrose, trehalose and raffinose, e.g. from sucrose and trehalose, e.g. trehalose; in an amount of about 200 mM, or an amino acid selected from arginine, glycine, histidine, tryptophane and methionine, e.g. arginine and methionine, e.g. methionine; e.g. in an amount of about 10 to 500 mM, e.g. in an amount of about 10 to about 300 mM, e.g. methionine in an amount of 10 mM, or in an amount of about 100 to about 300 mM; and the tonicity agent is selected from the group consisting of sodium chloride, amino acids and sugars, e.g. from trehalose and arginine.

Another aspect of invention relates to a pharmaceutical liquid formulation of an antibody against human OX40L comprising a stabilizer and a tonicity agent wherein the stabilizer is methionine in an amount of 10 mM; and the tonicity agent is selected from trehalose and arginine, e.g. in an amount of from about 100 to about 300 mM, e.g. trehalose in an amount of about 200 mM or arginine in an amount of about 130 mM.

In some embodiments, the pharmaceutical liquid formulation of an antibody against human OX40L comprises 20 to 200 mg/mL of an OX40L human antibody. In some embodiments, pharmaceutical liquid formulation of an antibody against human OX40L comprises 20 to 160 mg/mL, 100 to 160 mg/mL, 140 to 160 mg/mL, or 145 to 155 mg/mL. In one embodiment, the pharmaceutical liquid formulation of an antibody against human OX40L comprises approximately 150 mg/mL of an OX40L human antibody. In some embodiments, the antibody against human OX40L is a human or humanized antibody. In some embodiments, the antibody against human OX40L is a human antibody. In some embodiments, the antibody against human OX40L is a human antibody comprising as light chain the light chain of SEQ ID NO:1 and as heavy chain the heavy chain of SEQ ID NO:2 or 3.

One aspect of the invention relates to a pharmaceutical liquid formulation of an antibody against human OX40L comprising:

1 to 200 mg/ml of said antibody 20 mM of a buffer selected from histidine acetate or sodium acetate, e.g. histidine acetate in an amount of 20 mM;

0.001 to 1% of a surfactant;

(a) 5 to 500 mM of a stabilizer; or

(b) 5 to 500 mM of a stabilizer and 5 to 500 mM of a tonicity agent; or

(c) 5 to 500 mM of a tonicity agent;

at a pH in the range of 4.0-7.0.

In one embodiment, said antibody against human OX40L is a human antibody. In one embodiment, said antibody against human OX40L is a human antibody comprising the light chain of SEQ ID NO:1 and the heavy chain of SEQ ID NO:2 or 3.

One aspect of the invention relates to a pharmaceutical liquid formulation of an antibody against human OX40L comprising:

1 to 200 mg/ml of said antibody;

20 mM of a buffer selected from histidine acetate or sodium acetate, e.g. histidine acetate in an amount of 20 mM;

0.001 to 1% of a surfactant;

(a) 5 to 500 mM of a stabilizer; or

(b) 5 to 500 mM of a stabilizer and 5 to 500 mM of a tonicity agent; or

(c) 5 to 500 mM of a tonicity agent;

at a pH in the range of 5.0-6.0.

In one embodiment, said antibody against human OX40L is a human antibody. In one embodiment, said antibody against human OX40L is a human antibody comprising the light chain of SEQ ID NO:1 and the heavy chain of SEQ ID NO:2 or 3.

One aspect of the invention relates to a pharmaceutical liquid formulation of an antibody binding to human OX40L comprising:

1 to 200 mg/ml of said antibody; 1 to 100 mM of a buffer;

0.001 to 1% of a surfactant;

5 to 500 mM of a stabilizer and 5 to 500 mM of a tonicity agent, wherein the stabilizer is selected from the group consisting of sugars and amino acids; and the tonicity agent is selected from the group consisting of sodium chloride, amino acids and sugars, e.g. a sugar as stabilizer, e.g. selected from sucrose, trehalose and raffinose, e.g. from sucrose and trehalose, e.g. trehalose, in an amount of about 200 mM, or an amino acid as stabilizer selected from arginine, glycine, histidine, tryptophane and methionine, e.g. arginine and methionine, e.g. methionine, e.g. in an amount of about 10 to 500 mM, e.g. in an amount of about 10 to about 300 mM, e.g. methionine in an amount of 10 mM, or in an amount of about 100 to about 300 mM, e.g. arginine in an amount of about 130 mM; and the tonicity agent is selected from the group consisting of sodium chloride, amino acids and sugars; at a pH in the range of from 4.0 to 7.0. In one embodiment, said antibody against human OX40L is a human antibody. In one embodiment, said antibody against human OX40L is a human antibody comprising the light chain of SEQ ID NO:1 and the heavy chain of SEQ ID NO:2 or 3

Another aspect of the invention relates to a pharmaceutical liquid formulation of a human antibody binding to human OX40L comprising:

1 to 200 mg/ml of said antibody comprising as light chain the light chain of SEQ ID NO:1 and as heavy chain the heavy chain of SEQ ID NO:2 or 3;

1 to 100 mM of a buffer selected from sodium acetate-buffer at pH 5.3 and histidine acetate buffer at pH 5.8;

0.001 to 1% of a surfactant;

(a) 5 to 500 mM of a stabilizer selected from methionine, trehalose and arginine HCl; or

(b) 5 to 500 mM of methionine and 5 to 500 mM of a tonicity agent selected from trehalose and arginine HCl; or

(c) 5 to 500 mM of a tonicity agent selected from trehalose and arginine HCl.

Another aspect of the invention relates to a pharmaceutical liquid formulation of a human antibody binding to human OX40L comprising:

1 to 200 mg/ml of said antibody comprising as light chain the light chain of SEQ ID NO:1 and as heavy chain the heavy chain of SEQ ID NO:2 or 3;

20 mM of a buffer selected from sodium acetate-buffer at pH 5.3 and histidine acetate buffer at pH 5.8;

0.001 to 1% of a surfactant;

(a) a stabilizer selected from 10 mM methionine, 200 mM trehalose and 130 mM arginine HCl; or

(b) 10 mM methionine and a tonicity agent selected from 200 mM trehalose and 130 mM arginine HCl; or

(c) a tonicity agent selected from 200 mM trehalose and 130 mM arginine HCl.

Another aspect of the invention relates to a pharmaceutical liquid formulation of a human antibody binding to human OX40L comprising:

1 to 200 mg/ml of said antibody comprising as light chain the light chain of SEQ ID NO:1 and as heavy chain the heavy chain of SEQ ID NO:2 or 3;

20 mM of a buffer selected from sodium acetate-buffer at pH 5.3 and histidine acetate buffer at pH 5.8;

0.05% of a surfactant selected from Polysorbate 20, Polysorbate 80 and Poloxamer 188;

(a) a stabilizer selected from methionine, trehalose and arginine HCl; or

(b) methionine and a tonicity agent selected from trehalose and arginine HCl; or

(c) a tonicity agent selected from trehalose and arginine HCl.

Another aspect of the invention relates to a pharmaceutical liquid formulation of a human antibody binding to human OX40L comprising:

150 mg/ml of said antibody; and

(i) 20 mM sodium acetate-buffer at pH 5.3;

0.05% of Polysorbate 20;

mM methionine;

200 mM trehalose; or

(ii) 20 mM histidine acetate buffer at pH 5.8;

0.05% of Polysorbate 20; and

200 mM trehalose; or

(iii) 20 mM of histidine acetate buffer at pH 5.8;

0.05% of Polysorbate 80; and

200 mM trehalose; or

(iv) 20 mM of histidine acetate buffer at pH 5.8;

0.05% of Polysorbate 20; and

130 mM arginine HCl; or

(v) 20 mM of histidine acetate buffer at pH 5.8;

0.05% of Polysorbate 20;

mM methionine; and

200 mM trehalose; or

(vi) 20 mM of histidine acetate buffer at pH 5.8;

0.05% of Poloxamer 188;

mM methionine; and

200 mM trehalose.

In one embodiment, said antibody against human OX40L is a human antibody. In one embodiment, said antibody against human OX40L is a human antibody comprising as light chain the light chain of SEQ ID NO:1 and as heavy chain the heavy chain of SEQ ID NO:2 or 3. In one embodiment, said antibody against human OX40L is a human antibody comprising as light chain the light chain of SEQ ID NO:1 and as heavy chain the heavy chain of SEQ ID NO:2. In one embodiment, said antibody against human OX40L is a human antibody comprising as light chain the light chain of SEQ ID NO:1 and as heavy chain the heavy chain of SEQ ID NO:3.

The human monoclonal antibodies against OX40L may be produced by recombinant means, e.g. by those described in WO2006/029879. Such methods are widely known in the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate host cells like CHO cells, NS0 cells, SP2/0 cells, HEK293 cells, COS cells, and the antibody is recovered from the cells (supernatant or cells after lysis) by standard techniques, like column chromatography and others well known in the art, e.g. as described in WO2006/029879.

The formulation according to the invention can be prepared by methods known in the art, e.g. ultrafiltration-diafiltration, dialysis, addition and mixing, and combinations thereof. Examples of preparations of formulations according to the invention can be found hereinafter.

A composition of the present invention may be administered by a variety of methods known in the art. As will be appreciated by the skilled artisan, the route and/or mode of administration will vary depending upon the desired results.

To administer a composition of the invention by certain routes of administration, it may be necessary to dilute the composition in a diluent. Pharmaceutically acceptable diluents include saline, glucose, Ringer and aqueous buffer solutions.

The formulation according to the invention may be administered by intravenous (i.v.), subcutaneous (s.c.) or any other parenteral administration means such as those known in the pharmaceutical art.

The composition must be sterile and fluid to the extent that the composition is deliverable by parenteral administration, e.g., via a syringe needle or a cannula.

The formulations according to the invention are useful for prevention and/or treatment of inflammatory diseases in a mammal, e.g. a patient suspected of having or suffering from such a disease. Examples of such diseases include allergic reactions, e.g., asthma or allergic rhinitis

For example, the formulations of the present invention may be used for the treatment of allergic rhinitis or asthma, e.g. moderate asthma, moderate to severe asthma or persistent asthma in patients whose symptoms are not adequately controlled with inhaled corticosteroids. The patient population may include adults and adolescents (12 years of age and older). The formulations may be delivered, e.g., subcutaneously, e.g. once or twice a month.

In one embodiment, the formulation of the present invention may be supplied in 2 ml single use vials as e.g. 1.25 ml of a 150 mg/ml liquid for injection (1 ml extractable volume). An exemplary administration procedure may be as follows: The vial is allowed to warm up to room temperature. A transfer needle is attached to a disposable 1 ml syringe. The whole content of the vial is withdrawn into the syringe. The transfer needle is removed and an injection needle for subcutaneous administration is attached. The formulation of the present invention is administered subcutaneously, in the right or left arm/thigh, at room temperature. Main endpoint may, e.g., be decrease in acute exacerbations. Other endpoints include peak flow, daytime asthma symptoms, nocturnal awakenings, quality of life, emergency room visits, asthma free days, beta-2 agonist use, steroid reduction or tapering and effect on hyper-responsiveness.

In one embodiment, the liquid pharmaceutical formulation of the invention may be administered to a subject via a prefilled syringe, an autoinjector pen, or a needle-free administration device. Thus, another aspect of the invention provides an autoinjector pen, a prefilled syringe, or a needle-free administration device comprising the liquid pharmaceutical formulation of the invention. In one embodiment, the delivery device comprises a dose of the formulation comprising 20 to 200 mg/mL, 50 to 160 mg/mL, 100 to 160 mg/mL, 140 to 160 mg/mL, or 145 to 155 mg/mL of an antibody against human OX40L. In one embodiment, the delivery device comprises a dose of the formulation comprising approximately 150 mg/mL an antibody against human OX40L. In certain embodiments, the delivery device comprising the liquid pharmaceutical formulation of the invention comprises a dose of about 20 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg, or more of an antibody against human OX40L.

### EXAMPLES

The following Examples are examples of the formulations of the invention. It is understood that various other embodiments may be practiced, given the general description provided above.

### Example 1

### Preparation of Liquid Formulations

Mab1 was provided at a concentration of approx 20-40 mg/mL Mab1 in a 20 mM histidine buffer at a pH of approx. 5.5-6.0. Liquid formulations of Mab1 were prepared by diafiltration against a diafiltration buffer containing the anticipated buffer composition and where required by ultrafiltration to increase the Mab1 protein concentration to approx. 200 mg/mL. Excipients for stabilizing the antibody and for tonicity adjustment as well as surfactants were added as stock solutions to the antibody solution as required. Finally the Mab1 protein concentration was adjusted to the desired concentration by dilution with buffer to the final Mab1 concentration of approx. 20 mg/mL or 150 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approx. 2.4 mL.

### Liquid Formulations of 150 mg Mab/ml

| | Buffer | | surfactant | methionine | Excipient |
|---|---|---|---|---|---|
| | [20 mM] | pH | [%] | [mM] | [mM] |
| 1 | sodium acetate | 5.3 | 0.05 PS20 | 0 | 200 trehalose |
| 2 | sodium acetate | 5.3 | 0.05 PX188 | 0 | 200 trehalose |
| 3 | sodium acetate | 5.3 | 0.05 PS80 | 0 | 200 trehalose |
| 4 | sodium acetate | 5.3 | 0.03 PX188 | 0 | 200 trehalose |
| 5 | sodium acetate | 5.3 | 0.05 PS20 | 0 | 130 arginine HCl |
| 6 | sodium acetate | 5.3 | 0.05 PX188 | 0 | 130 arginine HCl |
| 7 | sodium acetate | 5.3 | 0.05 PX188 | 10 | 200 trehalose |
| 8 | sodium acetate | 5.3 | 0.05 PS20 | 10 | 200 trehalose |
| 9 | histidine acetate | 5.8 | 0.05 PS20 | 0 | 200 trehalose |
| 10 | histidine acetate | 5.8 | 0.05 PX188 | 0 | 200 trehalose |
| 11 | histidine acetate | 5.8 | 0.05 PS80 | 0 | 200 trehalose |
| 12 | histidine acetate | 5.8 | 0.03 PX188 | 0 | 200 trehalose |
| 13 | histidine acetate | 5.8 | 0.05 PS20 | 0 | 130 arginine HCl |
| 14 | histidine acetate | 5.8 | 0.05 PX188 | 0 | 130 arginine HCl |
| 15 | histidine acetate | 5.8 | 0.05 PS20 | 10 | 200 trehalose |
| 16 | histidine acetate | 5.8 | 0.05 PX188 | 10 | 200 trehalose |

PS20: Polysorbate 20,
PX188: Poloxamer 188

### Example 2

### Stability of Liquid Formulations

The formulations according to Example 1 were stored at different ICH climate conditions (2 to 8° C. and 40° C.) for different intervals of time and analyzed by 1) UV spectrophotometry, and 2) Size Exclusion Chromatography (SEC).

Size Exclusion Chromatography (SEC) was used to detect soluble high molecular weight species (HMW aggregates) and low molecular weight hydrolysis products (LMW) in the formulations. Analysis was performed on a Water Alliance 2795 HPLC instrument equipped with a TSKgel G3000 SWXL column (7.8×300 mm) Intact monomer, aggregates and hydrolysis products were separated by an isocratic elution profile using 0.2M K₂HPO₄/0.25M KCL, pH 7.0 as mobile phase, and were detected at a wavelength of 280 nm UV spectroscopy, used for determination of protein content, was performed on a Varian Cary Bio UV spectrophotometer in a wavelength range from 240 nm to 400 nm Neat protein samples were diluted to approx. 0.5 mg/mL with the corresponding formulation buffer. The protein concentration was calculated according to equation 1.

$$Protein content = \frac{A ( 280 ) - A ( 320 ) \times dil . factor}{ɛ 〈 \frac{{cm}^{2}}{mg} 〉 \times d ( cm )} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient ε.

TABLE 1

| | Protein conc. | Size Exclusion - HPLC | | |
|---|---|---|---|---|
| Timepoint | (mg/mL) | HMW (%) | Monomer (%) | LMW (%) |

Formulation 15
Storage at 2-8° C.

| | | | | |
|---|---|---|---|---|
| Initial | 156.4 | 0.9 | 99.1 | 0 |
| 1 months | 155.4 | 1.1 | 98.9 | 0 |
| 3 months | 156.6 | 1.1 | 98.8 | 0.1 |
| 6 months | 156.4 | 1.2 | 98.7 | 0.1 |

Storage at 40° C.

| | | | | |
|---|---|---|---|---|
| Initial | 156.4 | 0.9 | 99.1 | 0 |
| 1 months | n/a | 2.2 | 94.7 | 3.2 |
| 3 months | n/a | 2.4 | 93.1 | 4.5 |
| 6 months | n/a | 3.3 | 89.0 | 7.7 |

Formulation 9
Storage at 2-8° C.

| | | | | |
|---|---|---|---|---|
| Initial | 156.1 | 1.0 | 99.0 | 0 |
| 1 months | 158.2 | 1.2 | 98.7 | 0.1 |
| 3 months | 155.3 | 1.3 | 98.6 | 0.1 |
| 6 months | 153.4 | 1.5 | 98.4 | 0.1 |

Storage at 40° C.

| | | | | |
|---|---|---|---|---|
| Initial | 156.1 | 1.0 | 99.0 | 0 |
| 1 months | n/a | 2.9 | 93.9 | 3.2 |
| 3 months | n/a | 3.4 | 92.2 | 4.4 |
| 6 months | n/a | 4.9 | 87.6 | 7.5 |

Formulation 13
Storage at 2-8° C.

| | | | | |
|---|---|---|---|---|
| Initial | 158.9 | 0.9 | 99.1 | 0 |
| 1 months | 155.4 | 0.9 | 99.1 | 0 |
| 3 months | 160.7 | 1.0 | 99.0 | 0 |
| 6 months | 157.8 | 1.0 | 98.1 | 0.1 |

Storage at 40° C.

| | | | | |
|---|---|---|---|---|
| Initial | 158.9 | 0.9 | 99.1 | 0 |
| 1 months | n/a | 2.0 | 96.2 | 1.8 |
| 3 months | n/a | 3.4 | 91.4 | 5.2 |
| 6 months | n/a | 6.1 | 84.4 | 9.6 |

Formulation 8
Storage at 2-8° C.

| | | | | |
|---|---|---|---|---|
| Initial | 159.1 | 0.7 | 99.3 | 0 |
| 2 months | 160.5 | 1.1 | 98.9 | 0 |
| 3 months | 162.2 | 1.1 | 98.9 | 0 |

Storage at 40° C.

| | | | | |
|---|---|---|---|---|
| Initial | 159.1 | 0.7 | 99.3 | 0 |
| 2 months | n/a | 2.3 | 94.5 | 3.2 |
| 3 months | n/a | 2.6 | 92.8 | 4.6 |

Formulation 11
Storage at 2-8° C.

| | | | | |
|---|---|---|---|---|
| Initial | 156.5 | 1.0 | 99.0 | 0 |
| 2 months | 156.5 | 1.3 | 98.7 | 0 |
| 3 months | 154.6 | 1.3 | 98.6 | 0.1 |

Storage at 40° C.

| | | | | |
|---|---|---|---|---|
| Initial | 156.5 | 1.0 | 99.0 | 0 |
| 2 months | n/a | 3.2 | 93.4 | 3.4 |
| 3 months | n/a | 3.6 | 91.7 | 4.7 |

Formulation 16
Storage at 2-8° C.

| | | | | |
|---|---|---|---|---|
| Initial | 155.5 | 0.9 | 99.1 | 0 |
| 2 months | 157.2 | 1.1 | 98.9 | 0 |
| 3 months | 156.9 | 1.1 | 98.8 | 0.1 |

Storage at 40° C.

| | | | | |
|---|---|---|---|---|
| Initial | 155.5 | 0.9 | 99.1 | 0 |
| 2 months | n/a | 2.1 | 94.7 | 3.2 |
| 3 months | n/a | 2.4 | 93.1 | 4.5 |

### Example 3

### Comparison Between Liquid OX40L Antibody Formulations

The following formulations were used:

Formulation A: 20 mg Liquid Formulation based on the lyophilized formulation disclosed in WO2009/141239, i.e. 20 mg/mL Mab1, 20 mM histidine/histidine HCl, 240 mM trehalose, 0.02% polysorbate 20, at pH 6.0
Formulation B: 150 mg Liquid Formulation based on the lyophilized formulation disclosed in WO2009/141239, i.e. 150 mg/mL Mab1, 20 mM histidine/histidine HCl, 240 mM trehalose, 0.02% polysorbate 20, at pH 6.0
Formulation C: Liquid Formulation of the present invention, i.e. 20 mg/mL Mab1, 20 mM histidine acetate, 200 mM trehalose, 10 mM methionine, 0.05% polysorbate 20, at pH 5.8
Formulation D: Liquid Formulation of the present invention, i.e. 150 mg/mL Mab1, 20 mM histidine acetate, 200 mM trehalose, 10 mM methionine, 0.05% polysorbate 20, at pH 5.8

| | Formulation A | | | | Formulation C | | | |
|---|---|---|---|---|---|---|---|---|
| | | Size Exclusion - HPLC | | | | Size Exclusion - HPLC | | |
| Timepoint | Protein* | HMW | Mono | LMW | Protein* | HMW | Mono | LMW |
| | Storage at 2-8° C. | | | | | | | |
| Initial | 19.7 | 0.5 | 99.4 | 0.1 | 21.7 | 0.5 | 99.4 | 0.1 |
| 7 months | n/a | 0.6 | 99.3 | 0.1 | n/a | 0.5 | 99.4 | 0.1 |
| | Storage at 40° C. | | | | | | | |
| Initial | 19.7 | 0.5 | 99.4 | 0.1 | 21.7 | 0.5 | 99.4 | 0.1 |
| 7 months | n/a | 1.9 | 87.7 | 10.4 | n/a | 0.8 | 88.8 | 10.4 |

| | Formulation B | | | | Formulation D | | | |
|---|---|---|---|---|---|---|---|---|
| | | Size Exclusion - HPLC | | | | Size Exclusion - HPLC | | |
| Timepoint | Protein* | HMW | Mono | LMW | Protein* | HMW | Mono | LMW |
| | Storage at 2-8° C. | | | | | | | |
| Initial | 159.9 | 1.0 | 98.9 | 0.1 | 160.5 | 1.0 | 98.9 | 0.1 |
| 7 months | n/a | 1.7 | 98.2 | 0.1 | n/a | 1.3 | 98.6 | 0.1 |
| | Storage at 40° C. | | | | | | | |
| Initial | 159.9 | 1.0 | 98.9 | 0.1 | 160.5 | 1.0 | 98.9 | 0.1 |
| 7 months | n/a | 6.5 | 84.2 | 9.3 | n/a | 4.4 | 86.1 | 9.5 |

*Protein (mg/mL);
**HMW: high molecular weight (%); Mono: Monomer (%); LMW: low molecular weight (%)

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A pharmaceutical liquid formulation comprising:
1 to 200 mg/mL of an antibody against OX40L;
1 to 100 mM of a buffer selected from histidine acetate and sodium acetate;
0.001 to 1% of a surfactant;
(a) 5 to 500 mM of a stabilizer; or
(b) 5 to 500 mM of a stabilizer and 5 to 500 mM of a tonicity agent; or
(c) 5 to 500 mM of a tonicity agent;
at a pH in the range of from 4.0 to 7.0.

2. The formulation according to claim 1 comprising:
1 to 100 mM of a buffer selected from histidine acetate buffer at pH 5.8 and sodium acetate-buffer at pH 5.3;
(a) 5 to 500 mM of a stabilizer selected from methionine, trehalose and arginine HCl; or
(b) 5 to 500 mM of methionine as a stabilizer and 5 to 500 mM of a tonicity agent selected from trehalose and arginine HCl; or
(c) 5 to 500 mM of a tonicity agent selected from trehalose and arginine HCl.

3. The formulation according to claim 2 comprising:
20 mM of a buffer selected from sodium acetate-buffer at pH 5.3 and histidine acetate buffer at pH 5.8;
(a) a stabilizer selected from 10 mM methionine, 200 mM trehalose and 130 mM arginine HCl; or
(b) 10 mM methionine as a stabilizer and a tonicity agent selected from 200 mM trehalose and 130 mM arginine HCl; or
(c) a tonicity agent selected from 200 mM trehalose and 130 mM arginine HCl.

4. The formulation according to claim 1 comprising a surfactant selected from the group consisting of polysorbate 20 (sold under the trademark Tween 20™), polysorbate 80 (sold under the trademark Tween 80™) and a polyethylene-polypropylene copolymer sold under the name Poloxamer 188™.

5. The formulation according to claim 1 comprising a stabilizer and a tonicity agent wherein the stabilizer is selected from the group consisting of sugars and amino acids, and the tonicity agent is selected from the group consisting of sodium chloride, amino acids and sugars.

6. The formulation according to claim 5 wherein the stabilizer is selected from the group consisting of sucrose, trehalose, raffinose, arginine, glycine, histidine, tryptophane and methionine, and the tonicity agent is selected from the group consisting of trehalose and arginine.

7. The formulation according to claim 6 wherein the stabilizer is methionine in an amount of 10 mM; and the tonicity agent is selected from the group consisting of trehalose in an amount of about 200 mM and arginine in an amount of about 130 mM.

8. The formulation according to claim 1 comprising a tonicity agent selected from the group consisting of amino acids and sugars.

9. The formulation according to claim 8 wherein the tonicity agent is trehalose in an amount of 200 mM.

10. The formulation according to claim 8 wherein the tonicity agent is arginine in an amount of 130 mM.

11. The formulation according to claim 1, comprising histidine acetate in an amount of 20 mM, at pH 5.8.

12. The formulation according to claim 1, comprising sodium acetate in an amount of 20 mM, at pH 5.3.

13. The formulation according to according to claim 1 wherein the antibody against OX40L is a human antibody against human OX40L.

14. The formulation according to claim 13 wherein the antibody against OX40L is a human antibody against human OX40L comprising the light chain of SEQ ID NO:1 and the heavy chain of SEQ ID NO:2 or 3.

15. A method for the therapeutic and/or prophylactic treatment of an inflammatory disease, particularly for the treatment of asthma, which method comprises administering a liquid formulation according to claim 1 to a patient in need thereof.

16. A pharmaceutical liquid formulation comprising:
150 mg/ml of a human antibody against human OX40L comprising the light chain of SEQ ID NO:1 and the heavy chain of SEQ ID NO:2 or 3; and
(i) 20 mM sodium acetate-buffer at pH 5.3;
0.05% of a surfactant;
10 mM methionine; and
200 mM trehalose; or
(ii) 20 mM of histidine acetate buffer at pH 5.8;
0.05% of a surfactant;
10 mM methionine; and
200 mM trehalose.

17. The formulation of claim 16, wherein the surfactant is Polysorbate 20 or Poloxamer 188.

18. A method for the therapeutic and/or prophylactic treatment of an inflammatory disease, particularly for the treatment of asthma, which method comprises administering a liquid formulation according to claim 16 to a patient in need thereof.
